(19) United States
(12) Patent Application Publication
(10) Pub. No.: US 2006/0183692 A1
(43) Pub. Date: Aug. 17, 2006

(54) Use of low molecular weight thrombin inhibitors in cholesterol-lowering therapy

(76) Inventor: Margaretha Grind, Leicestershire (GB)

Correspondence Address: MORGAN LEWIS & BOCKIUS LLP, 1111 PENNSYLVANIA AVENUE NW, WASHINGTON, DC, 20004, US

(21) Appl. No.: 10/550,154

(22) PCT Filed: Mar. 19, 2004
(86) PCT No.: PCT/SE04/00417

§ 371 Date: Sep. 20, 2005

(30) Foreign Application Priority Data
Mar. 22, 2003 (GB) 0306615.6

(51) Int. Cl. A61K 38/04

(52) U.S. Cl. 514/19

(57) ABSTRACT

According to the invention there is provided a cholesterol lowering therapy method and a method for modification of lipid (triglyceride), lipoprotein, and apolipoprotein profiles associated with an increased risk of cardiovascular complications comprising the administration of a low molecular weight thrombin inhibitor, or a pharmaceutically acceptable derivative thereof, to a patient in need thereof.

### FIELD OF THE INVENTION

This invention relates to a new use of low molecular weight thrombin inhibitors.

### BACKGROUND AND PRIOR ART

It is well known that high levels of cholesterol are associated with heart disease. More than half of all US citizens are understood to have cholesterol levels that exceed those recommended, and one in five has cholesterol levels that are considered high.

Cholesterol is involved in the production and maintenance of cell membranes, as well as the production of sex hormones (including progesterone, testosterone, estradiol and cortisol), bile salts and Vitamin D. It is formed primarily in the liver, but also in other parts of the body, such as the small intestine.

In healthy individuals, all of the cholesterol that is needed to perform the above-mentioned functions is produced naturally. However, in a typical blood test, of the amount of cholesterol circulating in blood, about 85% is endogenous, the other 15% arising from external sources. Dietary cholesterol usually originates from meat, poultry, fish, seafood and dairy products. In this respect, high consumption levels of these foodstuffs may give rise to increased cholesterol levels in the bloodstream.

Increased cholesterol levels in serum have been associated with atherosclerosis, which is known to increase significantly the risk of blood vessel blockage (stenosis), and thus the likelihood of angina pectoris, myocardial infarction and other cardiovascular complications, such as stroke.

Cholesterol is insoluble in aqueous environments and thus needs to be transported within the bloodstream by apolipoproteins (Apos). When apolipoproteins are associated with cholesterol, complexes known as lipoproteins are formed. The density of these lipoproteins is determined by the amount of protein in the molecule and, in this respect, low-density lipoproteins (LDLs), which are the major cholesterol carrier in the blood, are known to have more of the negative effects mentioned herein than protective high-density lipoproteins (HDLs). High levels of LDLs are thus associated with atherosclerosis, whereas greater levels of HDLs are understood to provide some protection against stenosis, and hence coronary risk, by way of removal of excess cholesterol (transporting it to the liver for disposal).

A third group of carrier molecules, very low-density lipoproteins (VLDLs) are converted to LDLs following the delivery of triglycerides to the muscles and adipose tissue. Triglycerides are a mixture of fatty acids and glycerol and are the major components of lipids circulating in blood. Like cholesterol, triglycerides are substances that are found endogenously in the bloodstream, and may be deposited in adipose tissue. Triglycerides contain high-energy fatty acids which provide much of the fuel needed for normal cellular function. However, an excessive amount of triglycerides, or VLDLs, in the bloodstream can result in similar problems to those associated with high cholesterol and LDL levels, as well as obesity and diabetes.

Thus, levels of HDLs, LDLs, total cholesterol and triglycerides are all key indicators in determining the risk of atherosclerosis and associated cardiovascular disorders, such as coronary artery diseases (e.g. angina pectoris, myocardial infarction, etc.), stroke (including cerebro-vascular accident and transient ischaemic attack), peripheral arterial occlusive disease, obesity and diabetes. Patients with high overall cholesterol and/or triglycerides levels are at a significant risk, irrespective of whether or not they also have a favourable HDL level. Patients with normal cholesterol levels but low HDL levels are also at increased risk. Recently, it has also been noted that the level of risk of cardiovascular disease associated with high levels of apolipoprotein B (ApoB; which carries lipids in VLDLs and LDLs), and/or low levels of apolipoprotein A-I (ApoA-I; which carries lipids in HDLs), is extremely high.

There are numerous factors that influence cholesterol and triglyceride levels, including diet, age, weight, gender, genetics, diseases (such as diabetes) and lifestyle.

Positive changes in relation to diet, lifestyle and exercise are often insufficient to decrease the risk of cardiovascular problems. In such instances, cholesterol- and/or triglyceride-lowering medication may be prescribed.

Drugs that reduce LDL levels in serum can prevent or reduce the build-up of artery blocking plaques, and can reduce the risk of plaque rupture and associated thrombo-embolic complications. There are several types of drugs that can help reduce blood cholesterol levels. The most commonly prescribed are the statins, HMG-CoA reductase inhibitors, such as lovastatin, pravastatin, fluvastatin, simvastatin, atorvastatin, pitavastatin and rosuvastatin (e.g. rosuvastatin-calcium). These drugs prevent directly the formation of cholesterol in the liver and thus reduce the risk of cardiovascular disease. Other prescribed drug categories include resins (such as cholestyramine and colestipol), which act by binding bile acids, so causing the liver to produce more of the latter, and using up cholesterol in the process. Further, the B vitamin niacin has been reported at high doses to lower triglycerides and LDL levels in addition to increasing HDL levels. Fibrates (such as gemfibrozil and fenofibrate) are known to lower triglycerides and can increase HDL levels.

However, some of these drugs are known to have side effects, including liver damage. Hence, there is a need for alternative and/or more effective drugs for use in cholesterol-lowering therapy.

The early development of low molecular weight inhibitors of thrombin has been described by Claesson in Blood Coagul. Fibrinol. (1994) 5, 411. Low molecular weight thrombin inhibitors (and prodrugs thereof) have been described more recently in U.S. Pat. No. 4,346,078; International Patent Applications WO 93/11152, WO 93/18060, WO 93/05069, WO 94/20467, WO 94/29336, WO 95/35309, WO 95/23609, WO 96/03374, WO 96/06832, WO 96/06849, WO 96/17860, WO 96/24609, WO 96/25426, WO 96/32110, WO 97/01338, WO 97/02284, WO 97/15190, WO 97/23499, WO 97/30708, WO 97/40024, WO 97/46577, WO 98/06740, WO 97/49404, WO 97/11693, WO 97/24135, WO 97/47299, WO 98/01422, WO 98/57932, WO 99/29664, WO 98/06741, WO 99/37668, WO 99/37611, WO 98/37075, WO 99/00371, WO 99/28297, WO 99/29670, WO 99/40072, WO 99/54313, WO 96/31504, WO 00/01704, WO 00/08014, WO 00/35859, WO 00/35869, WO 00/42059, WO 00/61577, WO 00/61608, WO 00/61609, WO 01/87879, WO 02/14270, WO 02/44145, WO 03/000653, WO 04/000818 and WO 04/014894; and European Patent Applications 648 780, 468 231, 559 046, 641 779, 185 390, 526 877, 542 525, 195 212, 362 002, 364 344, 530 167, 293 881, 686 642, 669 317, 601 459 and 623 596.

In particular, international patent application WO 94/29336 discloses a group of compounds, including HOOC—CH₂—(R)Cgl-(S)Aze-Pab-H (in which Cgl represents cyclohexylglycyl, Aze represents azetidine-2-carboxyl and Pab-H represents 4-amidinobenzylamino), which is also known as melagatran (see Example 1 of WO 94/29336). International Patent Application WO 97/23499 discloses prodrugs of inter alia melagatran.

More recently, international patent application WO 02/44145 discloses α-hydroxy acid-based low molecular weight thrombin inhibitors and prodrugs thereof.

To the applicant's knowledge, none of the above-mentioned documents disclose or suggest the direct use a low molecular weight thrombin inhibitor or a prodrug thereof in cholesterol-lowering therapy and/or modifications of lipid (including triglyceride), lipoprotein, or apolipoprotein, profiles.

### DISCLOSURE OF THE INVENTION

We have found, surprisingly, that administration of a low molecular weight thrombin inhibitor may give rise to reduced levels of lipids, such as total cholesterol, LDLs (i.e. LDL-cholesterol) and triglycerides in the bloodstream, in addition to increasing HDL (i.e. HDL-cholesterol) levels.

According to a first aspect of the invention there is provided the use of a low molecular weight thrombin inhibitor, or a pharmaceutically acceptable derivative thereof, for the manufacture of a medicament for use in cholesterol-lowering therapy.

When employed in the context of the present invention and disclosure, the term “cholesterol-lowering therapy” includes any therapy that results in beneficial modifications of serum profiles of total cholesterol, lipids (including triglycerides), lipoproteins or apolipoproteins, and will thus be understood to encompass the terms “lipid-modifying therapy” and “lipid-(and triglyceride-) lowering therapy”, as well as the treatment of hyperlipidaemias (i.e. the elevation of lipids in the bloodstream), including hypercholesterolaemia (high cholesterol levels in the blood; including primary and secondary (combined) hypercholesterolaemia), hyperlipoproteinemia (elevated plasma lipoproteins levels) and/or hypertriglyceridemia (high triglyceride levels in the blood). The term will thus be understood to include types I, II (IIa and IIb), III, IV and/or V hyperlipoproteinaemia, as well as secondary hypemmiglyceridaemia and/or familial lecithin cholesterol acyltransferase deficiency, but in principle includes any treatment of a patient which results in a decrease in serum levels of cholesterol, LDLs, VLDLs, triglycerides and/or ApoB, and/or an increase in serum levels of HDLs and/or ApoA-I.

According to a second aspect of the invention there is provided a cholesterol-lowering therapy method, which method comprises the administration of a low molecular weight thrombin inhibitor, or a pharmaceutically acceptable derivative thereof, to a patient in need of such therapy.

For the avoidance of doubt, in the context of this disclosure, the terms “treatment”, “therapy” and “therapy method” include the therapeutic and/or prophylactic treatment of patients in need of modifications of cholesterol, lipid (including triglyceride), lipoprotein and/or apolipoprotein profiles.

“Pharmaceutically acceptable derivatives” of thrombin inhibitors includes salts (e.g. pharmaceutically-acceptable non-toxic organic or inorganic acid addition salts) and solvates. It will be appreciated that the term further includes derivatives that have, or provide for, the same biological function and/or activity as any relevant inhibitor. Thus, for the purposes of this invention, the term also includes prodrugs of thrombin inhibitors.

The term “low molecular weight thrombin inhibitor” will be understood by those skilled in the art. The term may also be understood to include any composition of matter (e.g. chemical compound) which inhibits thrombin to an experimentally determinable degree in in vivo and/or in in vitro tests, and which possesses a molecular weight of below 2,000, preferably below 1,000.

Preferred low molecular weight thrombin inhibitors include low molecular weight peptide-based, amino acid-based, and/or peptide analogue-based, thrombin inhibitors.

The term “low molecular weight peptide-based, amino acid-based, and/or peptide analogue-based, thrombin inhibitors” will be well understood by one skilled in the art to include low molecular weight thrombin inhibitors with one to four peptide linkages, and includes those described in the review paper by Claesson in Blood Coagul. Fibrin. (1994) 5, 411, as well as those disclosed in U.S. Pat. No. 4,346,078; International Patent Applications WO 93/11152, WO 93/18060, WO 93/05069, WO 94/20467, WO 94/29336, WO 95/35309, WO 95/23609, WO 96/03374, WO 96/06832, WO 96/06849, WO 96/17860, WO 96/24609, WO 96/25426, WO 96/32110, WO 97/01338, WO 97/02284, WO 97/15190, WO 97/30708, WO 97/40024, WO 97/46577, WO 98/06740, WO 97/49404, WO 97/11693, WO 97/24135, WO 97/47299, WO 98/01422, WO 98/57932, WO 99/29664, WO 98/06741, WO 99/37668, WO 99/37611, WO 98/37075, WO 99/00371, WO 99/28297, WO 99/29670, WO 99/40072, WO 99/54313, WO 96/31504, WO 00/01704, WO 00/08014, WO 00/35859, WO 00/35869, WO 00/42059, WO 00/61577, WO 00/61608, WO 00/61609, WO 01/87879, WO 02/14270, WO 02/44145, WO 03/000653 and WO 04/000818; and European Patent Applications 648 780, 468 231, 559 046, 641 779, 185 390, 526 877, 542 525, 195 212, 362 002, 364 344, 530 167, 293 881, 686 642, 669 317, 601 459 and 623 596, the specific and generic disclosures in all of which documents are hereby incorporated by reference.

Preferred low molecular weight peptide-based thrombin inhibitors include those described generically and specifically in international patent application WO 98/37075, including the compound that is the subject of claim 8 of that application as published (1-methyl-2-[N-(4-amidinophenyl)aminomethyl]-benzimidazol-5-yl-carboxylic acid, N-(2-pyridyl)-N-(2-hydroxycarbonyl-ethyl)amide) and prodrugs thereof, such as the compound that is the subject of claim 10 of that application as published (1-methyl-2-[N-[4-(N-n-hexyloxycarbonylamidino)phenyl]aminomethyl]benzimidazol-5-yl-carboxylic acid, N-(2-pyridyl)-N-(2-ethoxycarbonylethyl)amide) and those described in international patent application WO 04/014894.

Preferred low molecular weight peptide-based thrombin inhibitors also include HOOC—CH₂—(R)Cha-Pic-Nag-H (wherein Cha represents cyclohexylalanine, Pic represents (S)-pipecolinic acid and Nag represents noragmatine; known as inogatran; see International Patent Application WO 93/11152) and, especially, HOOC—CH₂—(R)Cgl-(S)Aze-Pab-H (known as melagatran; see above and International Patent Application WO 94/29336).

Further thrombin inhibitors include those of the formula I, 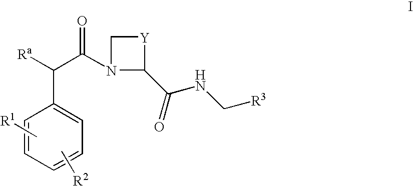
wherein
R^{a} represents —OH or —CH₂OH;
R¹ represents at least one optional halo substituent;
R² represents one or two C₁₋₃ alkoxy substituents, the alkyl parts of which substituents are themselves substituted with one or more fluoro substituents (i.e. R² represents one or two fluoroalkoxy(C₁-3) groups);
Y represents —CH₂— or —(CH₂)₂—; and
R³ represents a structural fragment of formula I(i) or I(ii): 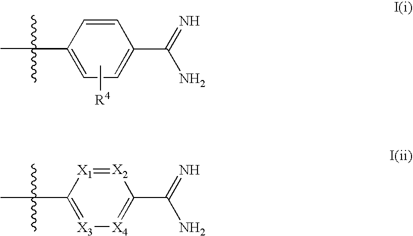
wherein
R⁴ represents H or one or more fluoro substituents; and
one or two of X₁, X₂, X₃ and X₄ represent —N— and the others represent —CH—.

Preferred compounds of formula I include:
(a) Ph(3-Cl)(5-OCHF₂)—(R)CH(OH)C(O)-(S)Aze-Pab: 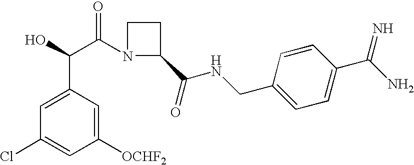
(b) Ph(3-Cl)(5-OCHF₂)—(R)CH(OH)C(O)—(S)Aze-Pab(2,6-diF): 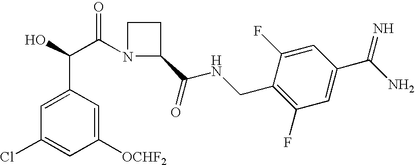
(c) Ph(3-Cl)(5-OCH₂CH₂F)—(R)CH(OH)C(O)—(S)Aze-Pab:

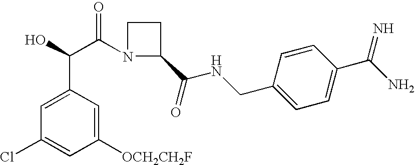

The term “prodrug” of a low molecular weight thrombin inhibitor includes any compound that, following oral or parenteral administration, is metabolised in vivo to form a low molecular weight thrombin inhibitor (as defined herein), in an experimentally-detectable amount, and within a predetermined time (e.g. within a dosing interval of between 6 and 24 hours (i.e. once to four times daily)). For the avoidance of doubt, the term “parenteral” administration includes all forms of administration other than oral administration.

Prodrugs of the thrombin inhibitor melagatran that may be mentioned include those disclosed in international patent application WO 97/23499. Preferred prodrugs are those of the formula R¹O₂C—CH₂—(R)Cgl-(S)Aze-Pab-OH (see the list of abbreviations above or in WO 97/23499), wherein R¹ represents C₁₋₁₀ alkyl or benzyl, such as linear or branched C₁₋₆ alkyl (e.g. C₁₋₄ alkyl, especially methyl, propyl and, particularly, ethyl) and the OH group replaces one of the amidino hydrogens in Pab.

Preferred prodrugs of compounds of formula I that may be mentioned include those of formula Ia, 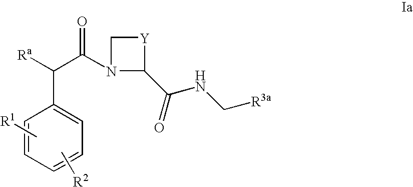
wherein R^{3a} represents a structural fragment of formula I(iii) or I(iv): 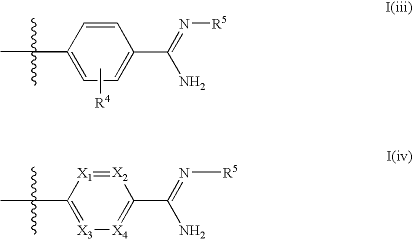
wherein R⁵ represents OR⁶ or C(O)OR⁷;
R⁶ represents H, C₁₋₁₀ alkyl, C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents);
R⁷ represents C₁₋₁₀ alkyl (which latter group is optionally interrupted by one or more oxygen atoms), or C₁₋₃ alkylaryl or C₁₋₃ alkyloxyaryl (the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents); and
R^{a}, R¹, R², Y, R⁴, X₁, X₂, X₃ and X⁴ are as hereinbefore defined.

Preferred prodrugs of compounds of formula I are methoxyamidine prodrugs thereof. Hence preferred compounds of formula Ia include:
(i) Ph(3-Cl)(5-OCHF₂)—(R)CH(OH)C(O)—(S)Aze-Pab(OMe): 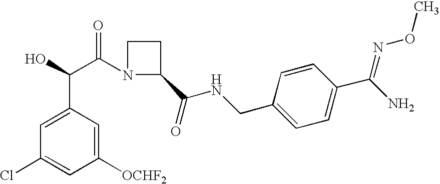
(ii) Ph(3-Cl)(5-OCHF₂)—(R)CH(OH)C(O)-(S)Aze-Pab(2,6-diF)(OMe): 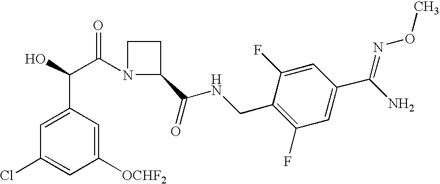
(iii) Ph(3-Cl)(5-OCH₂CH₂F)—(R)CH(OH)C(O)-(S)Aze-Pab(OMe):

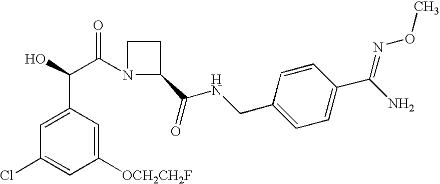

Compounds of formulae I and Ia may be made in accordance with techniques described in international patent application WO 02/44145.

In accordance with the invention, thrombin inhibitors and derivatives thereof may be administered orally, intravenously, subcutaneously, buccally, rectally, dermally, nasally, tracheally, bronchially, topically, by any other parenteral route, or via inhalation, in the form of a pharmaceutical preparation comprising the thrombin inhibitor or prodrug in a pharmaceutically acceptable dosage form. Depending on the disorder, and the patient, to be treated, as well as the route of administration, the compositions may be administered at varying doses.

Preferred modes of delivery are systemic. For melagatran and derivatives thereof, preferred modes of administration are parenteral, more preferably intravenous, and especially subcutaneous. For prodrugs of melagatran and compounds of formula Ia, preferred modes of administration are oral.

In the therapeutic treatment of manmals, and especially humans, thrombin inhibitors, prodrugs of thrombin inhibitors, and derivatives of either will generally be administered as a pharmaceutical formulation in admixture with a pharmaceutically acceptable adjuvant, diluent or carrier, which may be selected with due regard to the intended route of administration and standard pharmaceutical practice.

Suitable formulations for use in administering thrombin inhibitors are known in the art, and include those known from U.S. Pat. No. 4,346,078; International Patent Applications WO 93/11152, WO 93/18060, WO 93/05069, WO 94/20467, WO 94/29336, WO 95/35309, WO 95/23609, WO 96/03374, WO 96/06832, WO 96/06849, WO 96/17860, WO 96/24609, WO 96/25426, WO 96/32110, WO 97/01338, WO 97/02284, WO 97/15190, WO 97/30708, WO 97/40024, WO 97/46577, WO 98/06740, WO 97/49404, WO 97/11693, WO 97/24135, WO 97/47299, WO 98/01422, WO 98/57932, WO 99/29664, WO 98/06741, WO 99/37668, WO 99/37611, WO 98/37075, WO 99/00371, WO 99/28297, WO 99/29670, WO 99/40072, WO 99/54313, WO 96/31504, WO 00/01704, WO 00/08014, WO 00/35859, WO 00/35869, WO 00/42059, WO 00/61577, WO 00/61608, WO 00/61609, WO 01/87879, WO 02/14270, WO 02/44145, WO 03/000653 and WO 04/000818; and European Patent Applications 648 780, 468 231, 559 046, 641 779, 185 390, 526 877, 542 525, 195 212, 362 002, 364 344, 530 167, 293 881, 686 642, 669 317, 601 459 and 623 596, the disclosures in all of which documents are hereby incorporated by reference.

Suitable formulations for use with melagatran, derivatives and prodrugs thereof are described in the literature, for example as described in inter alia international patent applications WO 94/29336, WO 96/14084, WO 96/16671, WO 97/23499, WO 97/39770, WO 97/45138, WO 98/16252, WO 99/27912, WO 99/27913, WO 00/12043 and WO 00/13671, the disclosures in which documents are hereby incorporated by reference. Otherwise, the preparation of suitable formulations may be achieved non-inventively by the skilled person using routine techniques.

The amounts of thrombin inhibitor or derivative in the formulation will depend on the severity of the condition, and on the patient, to be treated, as well as the compound(s) which is/are employed, but may be determined non-inventively by the skilled person.

Suitable doses of thrombin inhibitors and derivatives thereof in the therapeutic and/or prophylactic treatment of mammalian, especially human, patients may be determined routinely by the medical practitioner or other skilled person, and include the respective doses discussed in the relevant prior art documents that are mentioned hereinbefore, the relevant disclosures in which are hereby incorporated by reference.

In the case of melagatran, suitable doses of active compound, prodrugs and derivatives thereof, in the therapeutic and/or prophylactic treatment of mammalian, especially human, patients include those which give a mean plasma concentration of up to 5 μmol/L, for example in the range 0.001 to 5 μmol/L over the course of treatment of the relevant condition. Suitable doses may thus be in the range 0.1 mg once daily to 25 mg three times daily, and/or up to 100 mg infused parenterally over a 24 hour period, for melagatran, and in the range 0.1 mg once daily to 100 mg three times daily for prodrugs of melagatran (see also the specific doses mentioned hereinafter for the prodrug of melagatran, ximelagatran).

In the case of compounds of formulae I and Ia, suitable daily doses of compounds in the therapeutic treatment of humans are about 0.001-100 mg/kg body weight at peroral administration and 0.001-50 mg/kg body weight at parenteral administration.

In any event, the physician, or the skilled person, will be able to determine the actual dosage which will be most suitable for an individual patient, which is likely to vary with the severity of the condition that is to be treated, as well as the age, weight, sex and response of the particular patient to be treated. The above-mentioned dosages are exemplary of the average case; there can, of course, be individual instances where higher or lower dosage ranges are merited, and such are within the scope of this invention.

Low molecular weight thrombin inhibitors may be employed in the method described herein by way of co-administration along with other cholesterol-lowering, or lipid-lowering/modifying, drugs/therapies that are mentioned hereinbefore, such as the statins (HMG-CoA reductase inhibitors) and particularly any one of those statins specifically mentioned hereinbefore, in combination therapy.

According to a further aspect of the invention, there is provided a combination product comprising:
(A) a low molecular weight thrombin inhibitor, or a pharmaceutically-acceptable derivative thereof; and
(B) another cholesterol-lowering, or lipid-lowering/modifying, therapeutic agent,
   wherein each of components (A) and (B) is formulated in admixture with a pharmaceutically-acceptable adjuvant, diluent or carrier.

Such combination products provide for the administration of low molecular weight thrombin inhibitor/derivative in conjunction with the other therapeutic agent, and may thus be presented either as separate formulations, wherein at least one of those formulations comprises thrombin inhibitor/derivative, and at least one comprises the other therapeutic agent, or may be presented (i.e. formulated) as a combined preparation (i.e. presented as a single formulation including thrombin inhibitor/derivative and the other therapeutic agent).

Thus, there is further provided:

(1) a pharmaceutical formulation including a low molecular weight thrombin inhibitor, or a pharmaceutically-acceptable derivative thereof; another cholesterol-lowering, or lipid lowering/modifying, therapeutic agent; and a pharmaceutically-acceptable adjuvant, diluent or carrier; and

(2) a kit of parts comprising components:

(a) a pharmaceutical formulation including a low molecular weight thrombin inhibitor, or a pharmaceutically-acceptable derivative thereof, in admixture with a pharmaceutically-acceptable adjuvant, diluent or carrier; and
(b) a pharmaceutical formulation including another cholesterol-lowering, or lipid lowering/modifying, therapeutic agent in admixture with a pharmaceutically-acceptable adjuvant, diluent or carrier,
   which components (a) and (b) are each provided in a form that is suitable for administration in conjunction with the other.

The method described herein may have the advantage that, in cholesterol-lowering therapy, it may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art for use in such therapy.

The invention is illustrated, but in no way limited, by the following examples, in which:

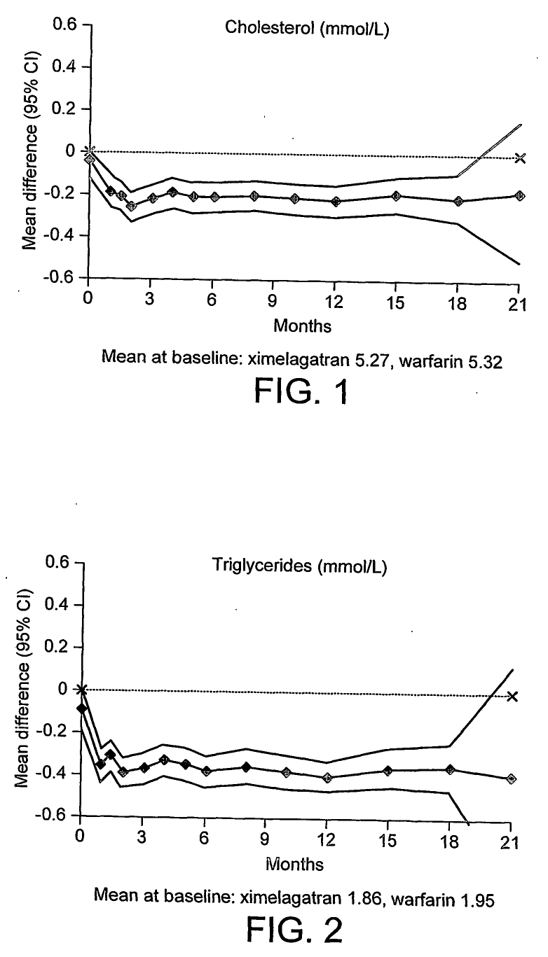

FIG. 1 illustrates the difference in mean values (with 95% Confidence Intervals) of total cholesterol levels in serum as between patients receiving ximelagatran (36 mg bid) or warfarin (INR levels between 2 and 3) during the course of a clinical trial over a 21 month period.

FIG. 2 illustrates the difference in mean values (with 95% Confidence Intervals) of levels of total triglycerides in serum as between patients receiving ximelagatran (36 mg bid) or warfarin (INR levels between 2 and 3) during the course of a clinical trial over a 21 month period.

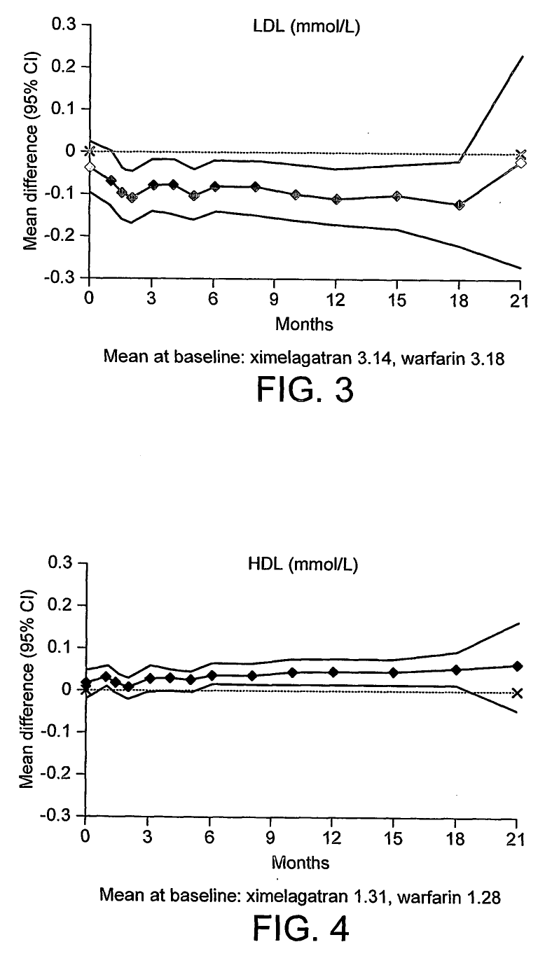

FIG. 3 illustrates the difference in mean values (with 95% Confidence Intervals) of LDL (i.e. LDL-cholesterol) levels in serum as between patients receiving ximelagatran (36 mg bid) or warfarin (INR levels between 2 and 3) during the course of a clinical trial over a 21 month period.

FIG. 4 illustrates the difference in mean values (with 95% Confidence Intervals) of HDL (i.e. HDL-cholesterol) levels in serum as between patients receiving ximelagatran (36 mg bid) or warfarin (INR levels between 2 and 3) during the course of a clinical trial over a 21 month period.

### EXAMPLE 1

Lipid Measurements in Patients Undergoing Thrombin Inhibition Therapy in a Clinical Trial

A large-scale Phase III clinical trial was set up to establish the efficacy of the study compound EtO₂C—CH₂—(R)Cgl-(S)Aze-Pab-OH (ximelagatran; see Example 17 of international patent application WO 97/23499) in the prevention of stroke in patients with non-valvular atrial fibrillation, as compared to the current frontline treatment for this indication, warfarin.

Ximelagatran is a prodrug of the low molecular weight thrombin inhibitor, melagatran (see Example 1 of international patent application WO 94/29226).

The clinical trial protocol was similar to that described in international patent application WO 02/36157, with the following major differences:
(a) the study objective was to show that the efficacy of ximelagatran is non-inferior to that of dose-adjusted warfarin, aiming for an INR 2.0-3.0 (with INR measurements taken at least every 28±3 days), in the prevention of all strokes (fatal and non-fatal) and systemic embolic events in patients with chronic non-valvular atrial fibrillation;
(b) the dosage of ximelagatran was fixed at 36 mg bid;
(c) in the exclusion criteria, subjects who had experienced stroke within the previous 30 days or transient ischaemic attack within the previous 3 days were excluded (as opposed to 2 years in the study described in WO 02/36157);
(d) the duration of treatment was long term (between 12 and 26 months); and
(e) the total number of patients in the trial was 3407 (as opposed to 220 in the study described in WO 02/36157). The study was a multicentre, multinational, IVRS-randomised, open-label, parallel-group study carried out across approximately 300 centres in approximately 25 countries.

Over the first 21 months of the trial, blood samples were taken from all patients in a standard manner at the following intervals: beforehand, and at, 1, 1.5, 2, 3, 4, 5, 6, 8, 10, 12, 15, 18, and 21, months. These samples were routinely analysed for total cholesterol, LDL (i.e. LDL-cholesterol), HDL (i.e. HDL-cholesterol), and total triglycerides, content using standard techniques for the detection of these lipids in serum.

A comparison was made between patients on ximelagatran (n=1704) and warfarin (n=1703). When the raw data were analysed, an unexpected statistically-significant difference in favour of the ximelagatran group was observed. As of the second month of treatment, marked mean differences were observed for cholesterol, triglycerides and LDL serum concentrations (consistently significantly lower in the ximelagtran group over the entire 21 month period), and for the HDL serum concentration (consistently significantly higher in the ximelagtran group over the entire 21 month period), as illustrated in FIGS. 1 to 4, respectively.

These data clearly demonstrate the potential utility of melagatran and derivatives thereof (e.g. prodrugs, such as ximelagtran), as well as, potentially, low molecular weight thrombin inhibitors and derivatives/prodrugs thereof, in cholesterol-lowering therapy.

1-13. (canceled)

14. A cholesterol-lowering therapy method, which method comprises the administration of melagatran inhibitor, or a pharmaceutically acceptable derivative thereof to a patient in need of such therapy.

15. (canceled)

16. The method of claim 14 wherein the therapy/treatment results in a decrease in serum levels of cholesterol, low-density lipoproteins, very low-density lipoproteins, triglycerides and/or apolipoprotein B; and/or an increase in serum levels of high-density lipoproteins and/or apolipoprotein A-I.

17. (canceled)

18. The method of claim 14, wherein the method comprises administering a prodrug of melagatran.

19. The method of claim 18, wherein the method comprises delivering a prodrug is of the formula:
R¹O₂C—CH₂—(R)Cgl-(S)Aze-Pab-OH,
wherein R¹ represents linear or branched C₁₋₆ alkyl and the OH group replaces one of the amidino hydrogens in Pab.

20. The method of claim 19, wherein R¹ represents methyl, ethyl, or propyl.

21. The method of claim 20, wherein R¹ represents ethyl.

22. A cholesterol-lowering therapy method, which method comprises the administration of a thrombin inhibitor of formula I, 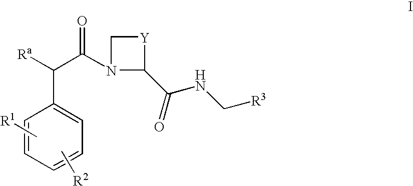
wherein
R^{a} represents —OH or —CH₂OH;
R¹ represents at least one optional halo substituent;
R² represents one or two C₁₋₃ alkoxy substituents, the alkyl parts of which substituents are themselves substituted with one or more fluoro substituents;
Y represents —CH₂— or —(CH₂)₂—; and
R³ represents a structural fragment of formula I(i) or I(ii): 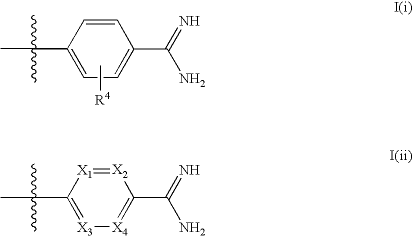
wherein
R⁴ represents H or one or more fluoro substituents; and
one or two of X₁, X₂, X₃, and X₄ represent —N— and the others represent —CH—, or a pharmaceutically acceptable derivative thereof, to a patient in need of such therapy.

23. The method of claim 22, wherein the thrombin inhibitor or derivative is:
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(2,6-diF); or
Ph(3-Cl)(5-OCH₂CH₂F)—(R)CH(OH)C(O)—(S)Aze-Pab.

24. The method of claim 22, wherein the the thrombin inhibitor or derivative is a prodrug of a thrombin inhibitor.

25. The method of claim 24, wherein the prodrug is of formula Ia, 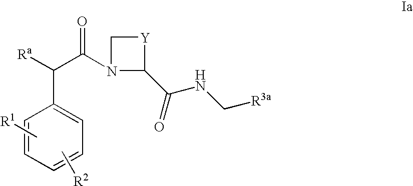
wherein R^{3a} represents a structural fragment of formula I(iii) or I(iv): 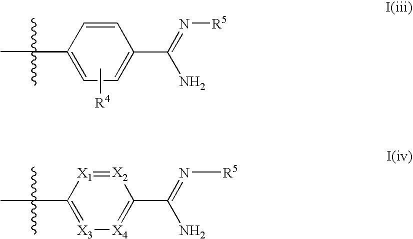
wherein R⁵ represents OR⁶ or C(O)OR⁷;
R⁶ represents H, C₁₋₁₀ alkyl, C₁₋₃ alkylaryl, or C₁₋₃ alkyloxyaryl, the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents; and
R⁷ represents C₁₋₁₀ alkyl, optionally interrupted by one or more oxygen atoms; C₁₋₃ alkylaryl; or C₁₋₃ alkyloxyaryl, the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents.

26. The method of claim 25, wherein the prodrug is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or
Ph(3-Cl)(5-OCH₂CH₂F)—(R)CH(OH)C(O)—(S)Aze-Pab(OMe).

27. A combination product comprising:
(A) a low molecular weight thrombin inhibitor, or a pharmaceutically-acceptable derivative thereof; and
(B) another cholesterol-lowering, or lipid-lowering/modifying, therapeutic agent,
wherein each of components (A) and (B) is formulated in admixture with a pharmaceutically-acceptable adjuvant, diluent or carrier.

28-29. (canceled)

30. The combination product of claim 27, wherein the thrombin inhibitor or derivative is melagatran.

31. A The combination product of claim 27, wherein the thrombin inhibitor or derivative is a prodrug of melagatran.

32. The combination product of claim 31, wherein the prodrug is of the formula:
R¹O₂C—CH₂—(R)Cgl-(S)Aze-Pab-OH,
wherein R¹ represents linear or branched C₁₋₆ alkyl and the OH group replaces one of the amidino hydrogens in Pab.

33. The combination product of claim 32, wherein R¹ represents methyl, ethyl, or propyl.

34. The combination product in of claim 33, wherein R¹ represents ethyl.

35. The combination product of claim 27, wherein the thrombin inhibitor or derivative is a compound of formula I, 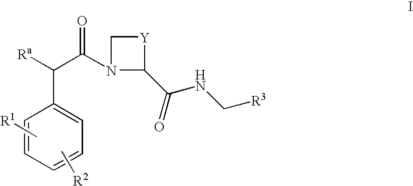
wherein
R^{a} represents —OH or —CH₂OH;
R¹ represents at least one optional halo substituent;
R² represents one or two C₁₋₃ alkoxy substituents, the alkyl parts of which substituents are themselves substituted with one or more fluoro substituents;
Y represents —CH₂— or —(CH₂)₂—; and
R³ represents a structural fragment of formula I(i) or I(ii): 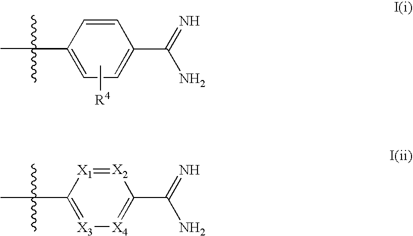
wherein
R⁴ represents H or one or more fluoro substituents; and
one or two of X₁, X₂, X₃, and X₄ represent —N— and the others represent —CH—.

36. The combination product of claim 27, wherein the thrombin inhibitor or derivative is:
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(2,6-diF); or
Ph(3-Cl)(5-OCH₂CH₂F)—(R)CH(OH)C(O)—(S)Aze-Pab.

37. The combination product of claim 27, wherein the thrombin inhibitor or derivative is a prodrug of a thrombin inhibitor.

38. The combination product of claim 27, wherein the thrombin inhibitor or derivative is a prodrug is of formula Ia, 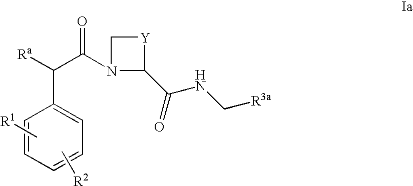
wherein R^{3a} represents a structural fragment of formula I(iii) or I(iv): 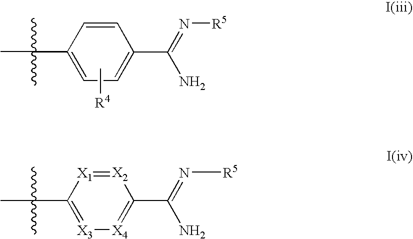
wherein R⁵ represents OR⁶ or C(O)OR⁷;
R⁶ represents H, C₁₋₁₀ alkyl, C₁₋₃ alkylaryl or C₁₋₃ alkyloxyaryl, the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents; and
R⁷ represents C₁₋₁₀ alkyl, which latter group is optionally interrupted by one or more oxygen atoms; C₁₋₃ alkylaryl; or C₁₋₃ alkyloxyaryl, the alkyl parts of which latter two groups are optionally interrupted by one or more oxygen atoms, and the aryl parts of which latter two groups are optionally substituted by one or more substituents selected from halo, phenyl, methyl or methoxy, which latter three groups are also optionally substituted by one or more halo substituents.

39. The combination product of claim 27, wherein the thrombin inhibitor or derivative is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or
Ph(3-Cl)(5-OCH₂CH₂F)—(R)CH(OH)C(O)—(S)Aze-Pab(OMe).

40. The combination product of claim 27, wherein the other therapeutic agent is a statin.

41. The combination product of claim 27, wherein the other therapeutic agent is lovastatin, pravastatin, fluvastatin, simvastatin, atorvastatin, pitavastatin, or rosuvastatin.

42-44. (canceled)

45. The method of claim 22, wherein the therapy/treatment results in a decrease in serum levels of cholesterol, low-density lipoproteins, very low-density lipoproteins, triglycerides and/or apolipoprotein B; and/or an increase in serum levels of high-density lipoproteins and/or apolipoprotein A-I.

46. The combination product of claim 27, wherein the thrombin inhibitor or derivative is of the formula:
R¹O₂C—CH₂—(R)Cgl-(S)Aze-Pab-OH,
wherein R¹ represents linear or branched C₁₋₆ alkyl and the OH group replaces one of the amidino hydrogens in Pab, and the other therapeutic agent is a statin.

47. The combination product of claim 27, wherein the thrombin inhibitor or derivative is of the formula:
R¹O₂C—CH₂—(R)Cgl-(S)Aze-Pab-OH,
wherein R¹ represents linear or branched C₁₋₆ alkyl and the OH group replaces one of the amidino hydrogens in Pab, and the other therapeutic agent is lovastatin, pravastatin, fluvastatin, simvastatin, atorvastatin, pitavastatin, or rosuvastatin.

48. The combination product of claim 27, wherein the thrombin inhibitor or derivative is of the formula:
EtO₂C—CH₂—(R)Cgl-(S)Aze-Pab-OH,
wherein Et represents ethyl and the OH group replaces one of the amidino hydrogens in Pab, and the other therapeutic agent is a statin

49. The combination product of claim 27, wherein the thrombin inhibitor or derivative is of the formula:
EtO₂C—CH₂—(R)Cgl-(S)Aze-Pab-OH,
wherein Et represents ethyl and the OH group replaces one of the amidino hydrogens in Pab, and the other therapeutic agent is lovastatin, pravastatin, fluvastatin, simvastatin, atorvastatin, pitavastatin, or rosuvastatin.

50. The combination product of claim 27, wherein the thrombin inhibitor or derivative is:
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(2,6-diF); or
Ph(3-Cl)(5-OCH₂CH₂F)—(R)CH(OH)C(O)—(S)Aze-Pab,
and the other therapeutic agent is a statin.

51. The combination product of claim 27, wherein the thrombin inhibitor or derivative is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or
Ph(3-Cl)(5-OCH₂CH₂F)—(R)CH(OH)C(O)—(S)Aze-Pab(OMe),
and the other therapeutic agent is a statin.

52. The combination product of claim 27, wherein the thrombin inhibitor or derivative is:
Ph(3-Cl)(5-OCHF₂)—(R)CH(OH)C(O)—(S)Aze-Pab;
Ph(3-Cl)(5-OCHF₂)—(R)CH(OH)C(O)—(S)Aze-Pab(2,6-diF); or
Ph(3-Cl)(5-OCH₂CH₂F)—(R)CH(OH)C(O)—(S)Aze-Pab,
and the other therapeutic agent is lovastatin, pravastatin, fluvastatin, simvastatin, atorvastatin, pitavastatin, or rosuvastatin.

53. The combination product of claim 27, wherein the thrombin inhibitor or derivative is:
Ph(3-Cl)(5-OCHF₂)—(R)CH(OH)C(O)—(S)Aze-Pab(OMe);
Ph(3-Cl)(5-OCHF₂)—(R)CH(OH)C(O)—(S)Aze-Pab(2,6-diF)(OMe); or
Ph(3-Cl)(5-OCH₂CH₂F)—(R)CH(OH)C(O)—(S)Aze-Pab(OMe),
and the other therapeutic agent is lovastatin, pravastatin, fluvastatin, simvastatin, atorvastatin, pitavastatin, or rosuvastatin.
